(19) United States
(12) Patent Application Publication
(10) Pub. No.: US 2018/0049400 A1
(43) Pub. Date: Feb. 22, 2018

(54) APPARATUS AND SYSTEM FOR A PET BED COVER CAPABLE OF RECEIVING A THERAPEUTIC PACKET

(71) Applicant: Lori Hills, Centerville, UT (US)

(72) Inventor: Lori Hills, Centerville, UT (US)

(21) Appl. No.: 15/801,236

(22) Filed: Nov. 1, 2017

Related U.S. Application Data
(63) Continuation-in-part of application No. 15/001,007, filed on Jan. 19, 2016.
(60) Provisional application No. 62/105,153, filed on Jan. 19, 2015.

(51) Int. Cl. A01K 1/035; A61D 9/00

(52) U.S. Cl. CPC A01K 1/0353; A61F 7/02; A61D 9/00

(57) ABSTRACT

An apparatus and system for a therapeutic pet bed cover are disclosed. The apparatus includes a durable top element that includes at least one pocket configured to receive a therapeutic packet; a flexible side element; and a bottom element configured to receive and retain a pet bed. The system includes a pet bed; a pet bed cover that includes a durable top element, a pocket configured to receive a therapeutic packet, a side element, and a bottom element configured to receive and retain a pet bed; and at least one therapeutic packet.

### CROSS-REFERENCE TO RELATED APPLICATIONS

The present application is a Continuation-in-Part Patent Application of and claims priority to U.S. patent application Ser. No. 15/001,007, filed on Jan. 19, 2016.

### BACKGROUND

### Field

This invention relates to pet bed covers and more particularly relates to a removable therapeutic cover and a system for a pet bed cover, a pet bed, and therapeutic packets.

### Description of the Related Art

Pet ownership has evolved over the past several decades to include greater concern for the health and comfort of a pet even when the pet is ill or aging. Veterinarians routinely treat ill and post-surgical animals that need special care for comfort and healing. In both situations, the animal can benefit from a comfortable bed that is configured to accommodate therapeutic elements. The pet owner and veterinarian can benefit from a bed with a cover that accommodates one or more therapeutic element and is also resistant to scratching and chewing as well as being washable and easy to change. Currently available pet beds lack a removable cover with a convenient housing for therapeutic elements.

### SUMMARY

From the foregoing discussion, it should be apparent that a need exists for a damage resistant pet bed cover that is durable, easy to change and wash and provides casing for therapeutic elements. Beneficially, such an apparatus, system, and method would be comfortable for the animal and allow one or more therapeutic elements to be positioned as desired.

The present invention has been developed in response to the present state of the art, and in particular, in response to the problems and needs in the art that have not yet been fully solved by currently available pet beds and pet bed covers. Accordingly, the present invention has been developed to provide a pet bed apparatus and system that overcome many or all of the above-discussed shortcomings in the art.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but does not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

Herein provided is a therapeutic pet bed cover comprising a durable top element having a pocket configured to receive a therapeutic packet. In some embodiments, the pet bed cover has a flexible side element and a bottom element configured to receive and retain a pet bed. The pet bed cover sometimes comprises a corner element configured to withstand chewing by an animal. In certain embodiments, the corner element further comprises a rigid material. In other embodiments, the corner element further comprises a Kevlar material. Yet other embodiments include a reinforcing element comprising Kevlar that reinforces the corner element. In various embodiments, the durable top element comprises a waterproof material.

The pocket of the pet bed cover may comprise a fastening element configured to close the pocket and retain the therapeutic packet. The pocket sometimes comprises at least one internal fastening element configured to removably attach to a therapeutic packet. In various embodiments, the internal fastening element comprises Velcro. In some embodiments, the pet bed cover comprises a plurality of pockets.

In certain embodiments, the bottom element of the pet bed comprises an elastic element configured to expand to receive a pet bed. The bottom element of the pet bed cover sometimes comprises a foldable flap and a fastener.

Further provided herein is a system for a therapeutic pet bed. In some embodiments, the system comprises a pet bed, a pet bed cover comprising a pocket configured to receive a therapeutic packet, and a therapeutic packet. The top element of the pet bed cover may be of durable material which in some embodiments is waterproof. The side element of the pet bed cover is sometimes flexible material. In certain embodiments, the bottom element of the pet bed cover is configured to receive and retain the pet bed. In various embodiments, the pocket further comprises a fastening element configured to close the pocket and retain the therapeutic packet.

In some embodiments of the system, corners of the pet bed are reinforced by Kevlar. In some embodiments of the system, the pet bed cover includes a corner element. In some embodiments, the corner element comprises Kevlar. In some embodiments, the corner element is reinforced by a reinforcing element that comprises Kevlar.

In some embodiments of the system provided, the pocket further comprises an internal fastening element configured to removably attach to the therapeutic packet. The therapeutic packet sometimes comprises a fastening element configured to removably attach to the pocket. In some embodiments, the systems further comprise a plurality of pockets. The therapeutic packet sometimes comprises at least one of a heat pack and a cold packet. The therapeutic packet may comprise medication and in certain embodiments aromatherapy. In various embodiments of system provided the pet bed comprises a compressible base of sufficient rigidity to fit snugly within the pet bed cover.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a perspective top view illustrating an embodiment of a pet bed cover in accordance with the present invention;

FIG. 2 is a perspective top view illustrating an embodiment of a system for a therapeutic pet bed in accordance with the present invention;

FIG. 3 is a bottom view illustrating an embodiment of a pet bed cover in accordance with the present invention;

FIG. 4 is a bottom view illustrating an embodiment of a pet bed cover in accordance with the present invention.

FIG. 5 is a bottom view illustrating an embodiment of a pet bed cover in accordance with the present invention.

FIG. 6 is a bottom view illustrating an embodiment of a pet bed cover in accordance with the present invention.

FIG. 7 is a top view illustrating an embodiment of a plurality of pockets in a pet bed cover;

FIG. 8 is a top view illustrating one embodiment of a system for a pet bed cover comprising a plurality of pockets and insertable therapeutic packets in accordance with the present invention;

FIG. 9 is a three-dimensional depiction illustrating an embodiment of a therapeutic pet bed cover installed on a pet bed and receiving a therapeutic packet in accordance with the present invention;

FIG. 10 is a close up three-dimensional depiction illustrating an embodiment of a therapeutic pet bed cover installed on a pet bed and receiving a therapeutic packet in accordance with the present invention; and

FIG. 11 is a three-dimensional depiction illustrating an embodiment of a pet bed and folded therapeutic pet bed covers.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, specific details are provided to facilitate a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

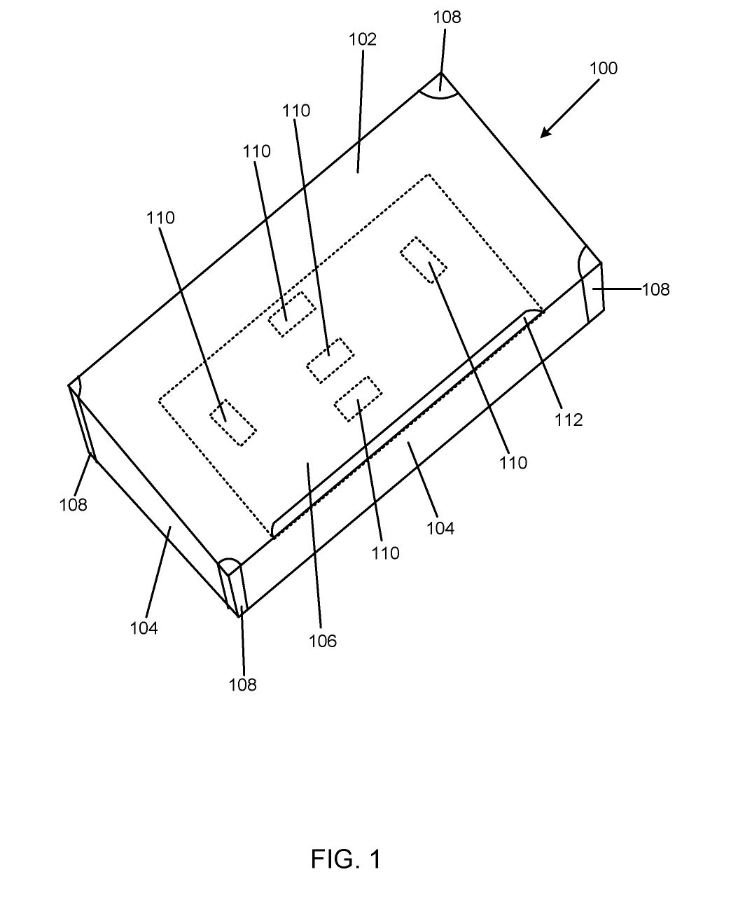

FIG. 1 depicts a perspective top view of an embodiment of a pet bed cover 100 in accordance with the present invention, comprising a durable top element 102, a side element 104, a therapeutic pocket 106, a chew resistant corner element 108, and an internal fastening element 110, (indicated by dotted lines), and a pocket sealing element 112. In some embodiments, the durable top element 102 is waterproof. In certain embodiments, the durable top element 102 is reinforced to be chew resistant and/or scratch resistant. In certain embodiments, the durable top element 102 is formed of a plush material, quilted top, or other similar material. In other embodiments, the durable top element 102 may comprise a lightweight material. In some embodiments, the side element 104 may comprise an elastic material such as Spandex™.

In some embodiments, the corner element 108 is rigid and may be configured to withstand chewing by an animal. The corner element 108 may comprise hard plastic, metal, fabric, or other sturdy material. In some embodiments, the corner element 108 may comprise a Kevlar material. In some embodiments, the corner element 108 is coupled to other elements by being sewn together with strong thread. In other embodiments, the corner element 108 may comprise both a rigid material and a Kevlar material. In yet further embodiments, the corner element 108 may not comprise Kevlar, but a reinforcing piece comprising a Kevlar material is disposed over each corner element and is coupled to other elements of the pet bed cover 100, such as the durable top element 102, the side element 104, bottom element 302 or other elements of the pet bed cover 100. In some embodiments, the reinforcing piece is coupled to other elements of the pet bed cover 100 by sewing with heavy-duty thread. In some embodiments, the reinforcing piece completely covers the corner element 108, and in other embodiments, the reinforcing piece partially covers the corner element 108. In further embodiments, the reinforcing piece completely covers the corner element 108, and partially covers other elements of the pet bed cover 100, such as the durable top element 102, the side element 104, the bottom element 302 or other elements of the pet bed cover 100.

In some embodiments, the internal fastening element 110 is located inside the therapeutic pocket 106 and may be on either or both of the upper and lower inside surfaces. The internal fastening element 110 is sometimes Velcro and may comprise a snap, a hook, a loop, or other device. In certain embodiments, the pocket sealing element 112 comprises Velcro. In some embodiments, the pocket sealing element 112 comprises a zipper. The pocket sealing element 112 may comprise a strip of rigid or semi-rigid material incorporated into the pocket edge. In some embodiments, the pocket sealing element 112 is the unimproved edge of the pocket.

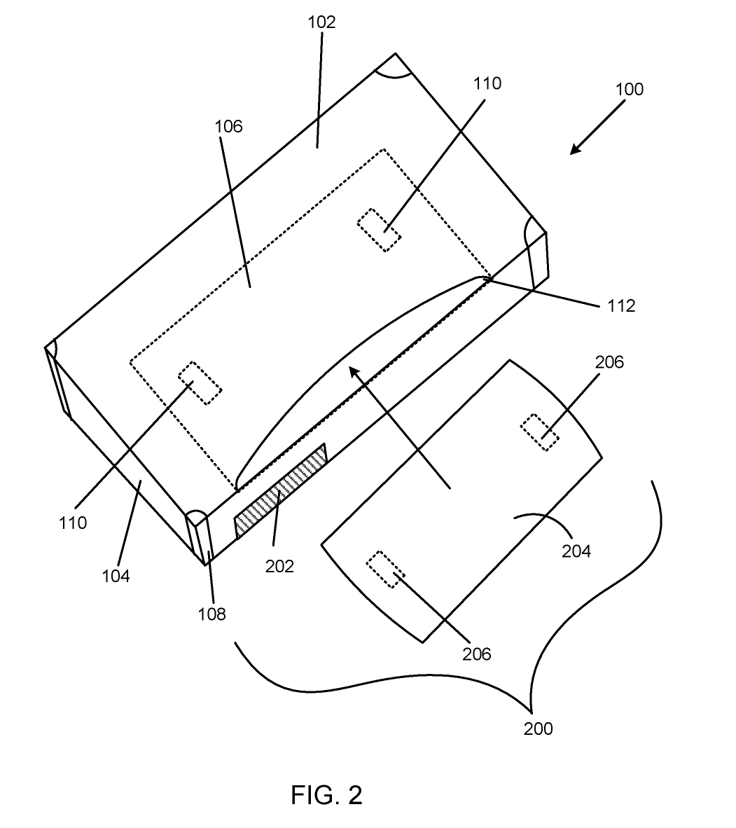

FIG. 2 depicts a perspective top and partial cut-away view of an embodiment of a system 200 for a therapeutic pet bed comprising a pet bed cover 100, a pet bed 202, a therapeutic pocket 106, and a therapeutic packet 204 in accordance with the present invention. In certain embodiments, the pet bed cover comprises a durable top element 102, a side element 104, a corner element 108, an internal fastening element 110, and a pocket sealing element 112. In some embodiments, the pocket sealing element 112 may close the opening to therapeutic pocket 106 and retain therapeutic packet 202 inside. The therapeutic packet 202 may further comprise a fastening element 204. The fastening element 204 may comprise Velcro, a snap, a hook, a loop, or other device and may be located on any surface of the therapeutic packet 202. In some embodiments, the fastening element 204 of the therapeutic packet 202 removably attaches to the internal fastening element 110 of the therapeutic pocket 106 to anchor the therapeutic packet 202. In certain embodiments, the therapeutic packet 202 comprises one or more of a heat pack, a cold pack, medication and aromatherapy.

In some embodiments, the pet bed 202 may comprise pet bed corner elements that comprise a Kevlar material. In other embodiments, the corners of the pet bed may be reinforced by pet bed corner reinforcing elements. In some embodiments, these pet bed corner reinforcing elements comprise a Kevlar material. A pet bed 202 with such features may be used in tandem with a pet bed cover 100 as shown in various embodiments to provide extra corner strength. In some cases, such a pet bed 202 may be used while the cover is being washed or is otherwise unavailable, and may still withstand wear and tear put on it by animals. In yet further embodiments, other portions or even the entire pet bed may also comprise a Kevlar material.

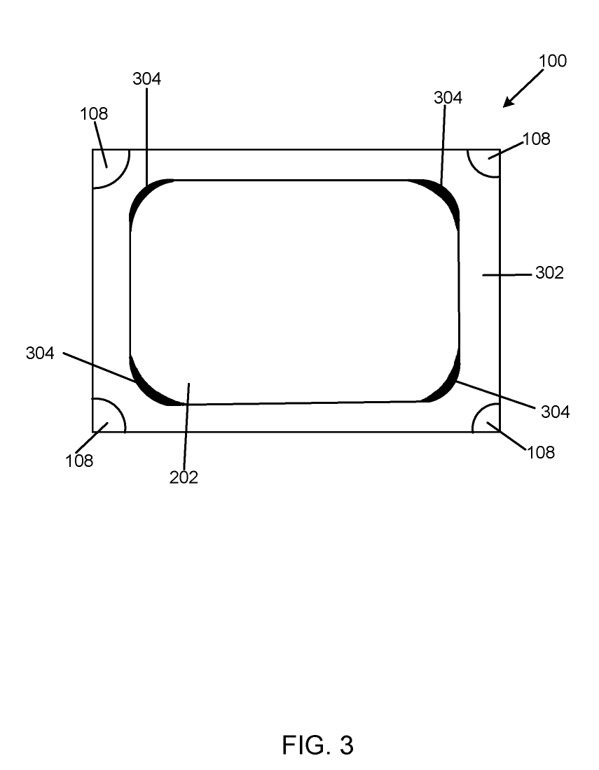

FIG. 3 depicts a bottom view of an embodiment of a pet bed cover 100 in accordance with the present invention, having a bottom element 302, corner elements 108 and expandable elements 304 configured to receive and retain a pet bed 202. In some embodiments, the expandable elements 304 comprise an elastic material. The elastic material may comprise the bottom element 302 itself or may comprise one, two, three, or four corners of the bottom element 302 of the pet bed cover 100.

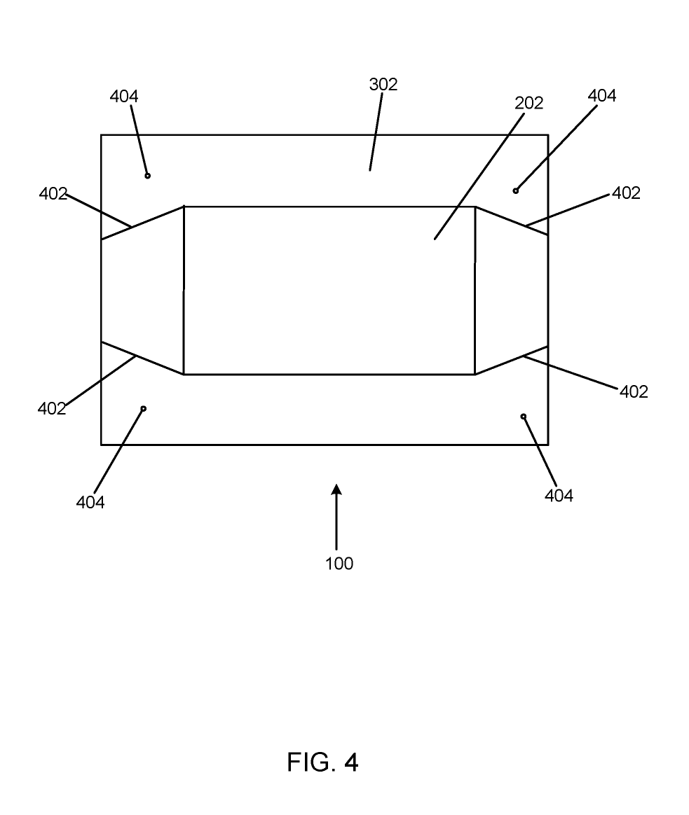

FIG. 4 depicts a bottom view of an embodiment of a pet bed cover 100 in accordance with the present invention having a foldable corner flap 402 and a fastener 404 configured to receive and retain a pet bed 202. In certain embodiments, the fastener 404 comprises a snap. In various embodiments, the fastener 404 comprises Velcro. The fastener 404 sometimes comprises a button, hook, hook and eye, zipper, rotatable insert or other device. The foldable corner flap 402 and fastener 404 may occupy one, two, three, or all of the corners of the bottom element 302 of the pet bed cover 100.

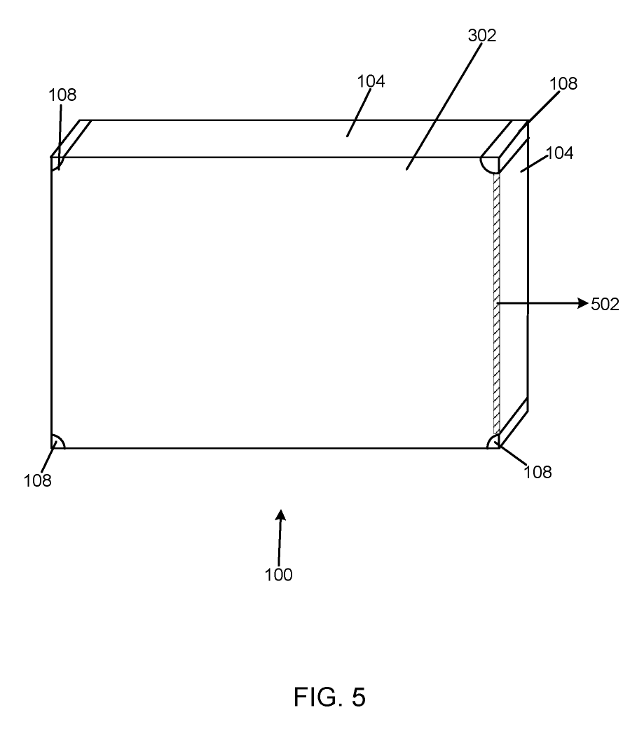

FIG. 5 depicts a perspective bottom view of a pet bed cover in accordance with the present invention, comprising a bottom element 302 and a cover closure element 502 as well as a side element 104 and corner elements 108. The cover closure element 502 is configured to allow the pet bed 202 to slide into the pet bed cover 100 and then to close the pet bed cover 100 to securely retain the pet bed 202. In some embodiments, the cover closure element 502 comprises one or more of a zipper, Velcro, a snap, a hook, or other device. The cover closure element 502 may be may be located in any side element 104 of the pet bed cover 100 and may be in any position on the side element 104. For example, the cover closure element 502 may be in the upper, lower, or either side extremity of the side element 104 and may be positioned vertically, horizontally, diagonally, or in any other configuration.

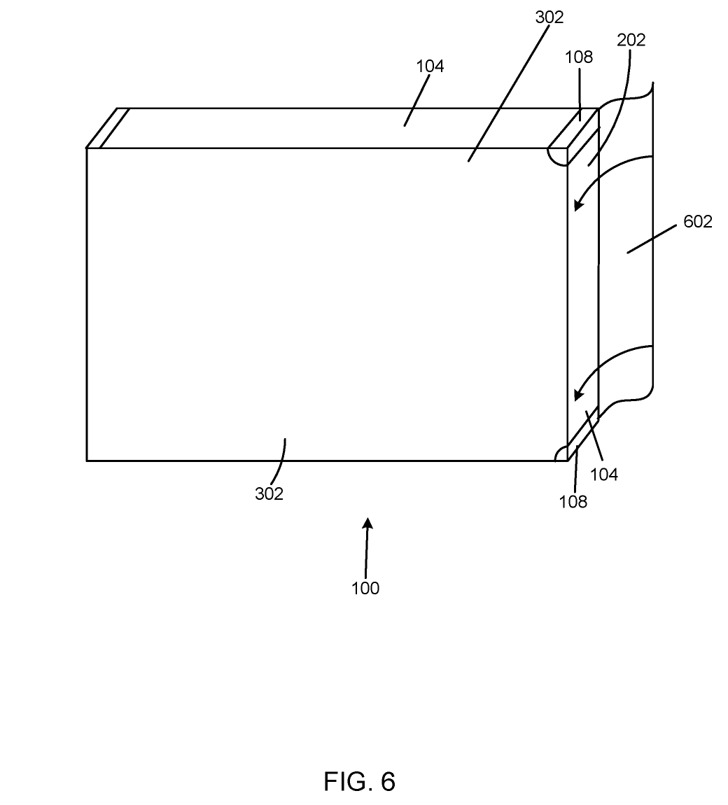

FIG. 6 depicts a perspective bottom view of a pet bed cover 100 in accordance with the present invention, comprising a bottom element 302, a side element 304, corner elements 108 and a closure flap 602. The closure flap 602 may comprise a long or short side of the pet bed cover 100 and may extend from the bottom, top, or side. After insertion of a pet bed 202 the closure flap may be tucked into either the bottom element 302 or the durable top element 102 of the pet bed 100. In some embodiments the closure flap 602 is fastened to the pet bed 100 with a closure device including without limitation Velcro, a zipper, a hook, a snap, a button, a hook and eye, and a loop.

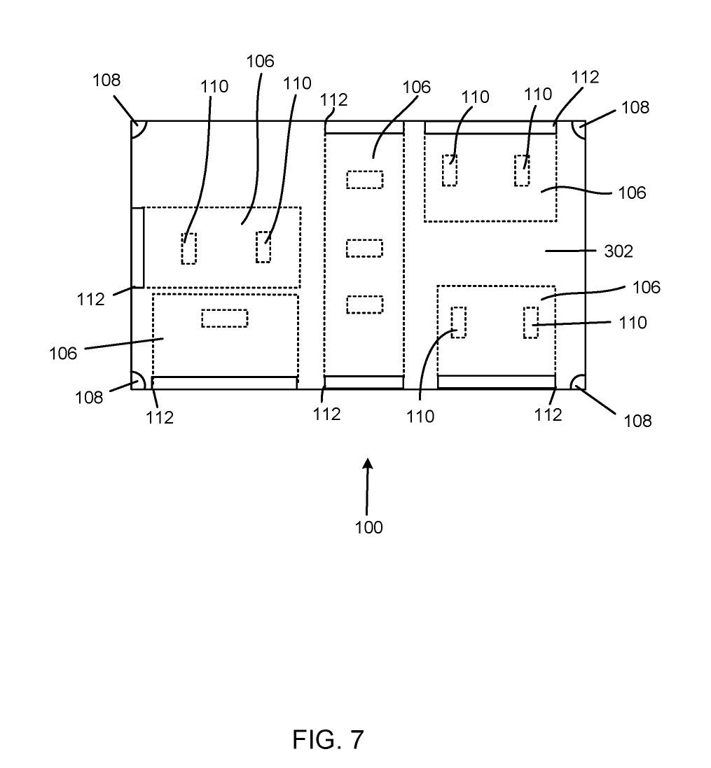

FIG. 7 depicts a top view of an embodiment of a pet bed cover 100 comprising a durable top element 102, a corner element 108, a plurality of therapeutic pockets 106, an internal fastening element 110 and a pocket sealing element 112. The durable pocket 106 may comprise any number of pockets and may be arranged in any position on the durable top element 102 of the pet bed 100. In therapeutic pocket 106 may be open on one or more sides. In some embodiments, the therapeutic pocket 106 may extend across the entire width or length of the pet bed cover 100 and may be open on one or more edges.

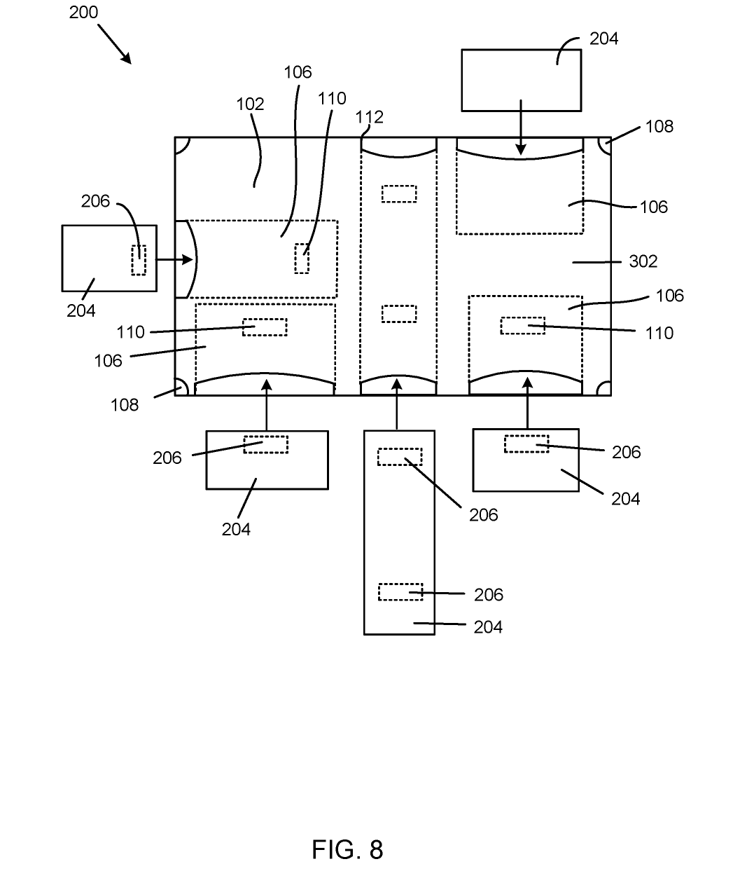

FIG. 8 depicts a top view of an embodiment of a system for a therapeutic pet bed comprising a durable top element 102, a plurality of therapeutic pockets 106 and a plurality of therapeutic pouches 204. In certain embodiments, the therapeutic pouches 204 may comprise one of more of a heat pack, a cold pack, medicine and aromatherapy. Any number of therapeutic pockets 106 may be arranged in any position on the durable top element 102 and any number of them may be occupied or unoccupied. Internal fastening elements 110 may interact with fastening elements 206 on the therapeutic pouches 204 to secure the therapeutic pouches 204 in position. The plurality of therapeutic pockets 106 may be open, closed, sealed or unsealed as shown in previous figures.

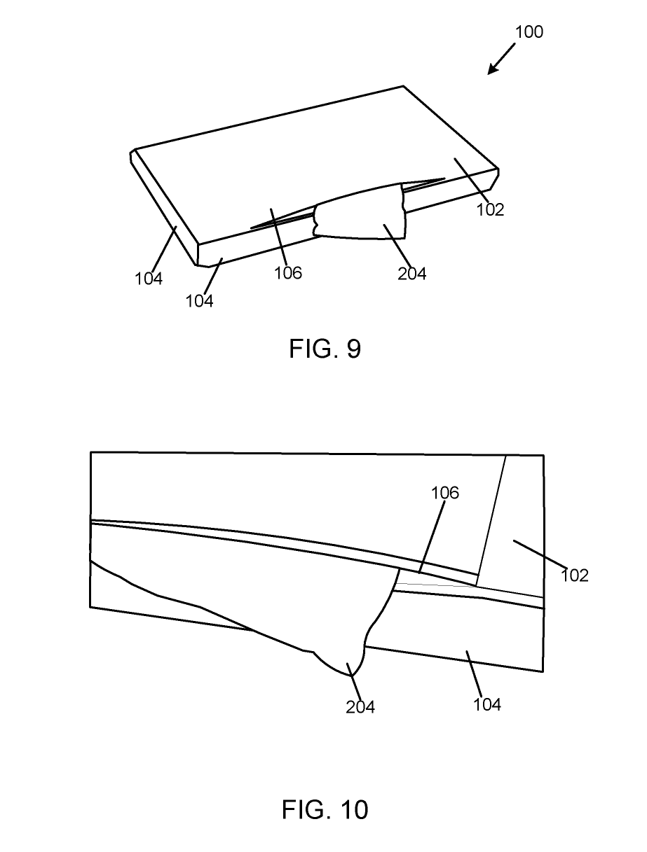

FIG. 9 is a three-dimensional illustration depicting an embodiment of a therapeutic pet bed cover 100 in accordance with the present invention, having a durable top element 102, a side element 104, and therapeutic pocket 106 receiving a therapeutic packet 204. In certain embodiments, the therapeutic packet 204 may be one or more of a cold pack, a heat pack, medication, and aroma therapy

FIG. 10 is a close up three-dimensional depiction illustrating an embodiment of a therapeutic pet bed cover 100 according to the present invention, having a durable top element 102, a side element 104 and a therapeutic pocket 106 receiving a therapeutic packet 204 in accordance with the present invention.

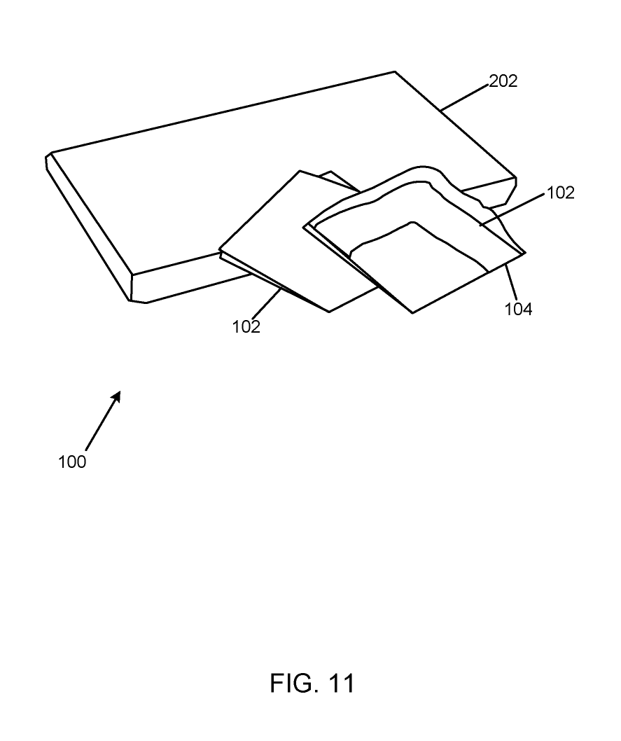

FIG. 11 is a three-dimensional illustration depicting an embodiment of a pet bed 202 and a folded therapeutic pet bed cover 100 with a durable top element 102 and a side element 104. It is to be understood that while the pet bed 202 and pet bed cover 100 depicted here are substantially rectangular the pet bed cover 100 and its elements as depicted and described in this and the preceding figures may be of any height and may assume any geometrical shape including square, triangular, circular, semicircular, spherical, hemispherical, and other geometrical forms.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A therapeutic pet bed cover, the pet bed cover comprising:
a durable top element further comprising at least one pocket configured to receive a therapeutic packet;
a flexible side element; and
a bottom element configured to receive and retain a pet bed.

2. The pet bed cover of claim 1, further comprising a corner element configured to withstand chewing by an animal.

3. The pet bed cover of claim 2, wherein the corner element comprises Kevlar.

4. The pet bed cover of claim 2, wherein
the corner element is reinforced with at least one reinforcing element comprising Kevlar.

5. The pet bed cover of claim 4, wherein
the reinforcing element is coupled to the corner element, the top element, the side element and the bottom element with thread.

6. The pet bed cover of claim 1 wherein the side element comprises Kevlar.

7. The pet bed cover of claim 1, wherein the at least one pocket comprises an edge fastening element configured to close the pocket and retain the therapeutic packet.

8. The pet bed cover of claim 1, wherein the at least one pocket comprises at least one internal fastening element configured to removably attach to the therapeutic packet.

9. The therapeutic pet bed cover of claim 1, wherein the bottom element further comprises an elastic element configured to expand to receive a pet bed.

10. The bottom element of claim 1, further comprising a foldable flap and a fastener.

11. A system for a therapeutic pet bed, the system comprising:
a pet bed;
a pet bed cover comprising
a durable top element,
a pocket configured to receive a therapeutic packet,
a flexible side element, and
a bottom element configured to receive and retain the pet bed; and
at least one therapeutic packet.

12. The system of claim 11, wherein at least one corner of the pet bed is reinforced with Kevlar.

13. The system of claim 11, wherein the pet bed cover further comprises a corner element.

14. The system of claim 13, wherein the corner element comprises Kevlar.

15. The system of claim 13, wherein the pet bed cover further comprises a reinforcing element that
comprises Kevlar; and
reinforces the corner element.

16. The system of claim 11, wherein the therapeutic packet further comprises a fastening element configured to removably attach to the pocket.

17. The system of claim 11, wherein the at least one therapeutic packet comprises at least one hot pack.

18. The system of claim 11, wherein the at least one therapeutic packet comprises at least one cold pack.

19. The system of claim 11, wherein the at least one therapeutic packet comprises medication.

20. The system of claim 11, wherein the at least one therapeutic packet comprises aromatherapy.
